(19) United States
(12) Patent Application Publication
(10) Pub. No.: US 2005/0042290 A1
(43) Pub. Date: Feb. 24, 2005

(54) Controlled release pharmaceutical formulation containing venlafaxine

(76) Inventors: Janez Kerc, Ljubljana (SI); Vlasta Humar, Stahovica (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/500,446

(22) PCT Filed: Mar. 1, 2002
(86) PCT No.: PCT/IB02/00001

(51) Int. Cl. A61K 9/24; A61K 31/277

(52) U.S. Cl. 424/471; 514/521

(57) ABSTRACT

A solid controlled release pharmaceutical formulation for once daily administration comprises a core comprising venlafaxine, polyvinylpyrrolidone, a low viscosity hydrophilic polymer and a high viscosity hydrophilic polymer, and a polymeric coating comprising a water high permeable polymer, and a water low permeable polymer. The invention further relates to a process for the preparation of a solid controlled release pharmaceutical formulation comprising the steps of dissolving venlafaxine and ployvinylpyrrolidone in an organic solvent, applying the resulting solution onto low viscosity polymer, homogeneously mixing the obtained granulate with a high viscosity polymer, and compressing the granulate to obtain a core which is then coated with a polymeric coating comprising a water high permeable polymer and a water low permeable polymer.

This invention relates to a controlled release pharmaceutical formulation for once daily administration, in particular to a controlled release pharmaceutical formulation of venlafaxine.

Venlafaxine, chemically named (±) 1-[2-(dimethylamino)-1(4-methoxyphenyl)ethyl]-cyclohexanol, is an antidepressant disclosed in EP-A-0 112 669. Presently venlafaxine hydrochloride is administered to adults as conventional immediate release tablets or as 24 hour extended-release multiparticulate capsules.

Venlafaxine hydrochloride is very soluble in water. It is known that it is very difficult to develop a pharmaceutical form with a very slow dissolution rate of freely soluble drug. Besides that, venlafaxine hydrochloride is polymorphic, so dissolution is dependent also on polymorphic form and particle size of particular polymorphic form. Therefore, it is a special task to develop such a pharmaceutical formulation that would sustain and control the dissolution of freely soluble drug over 24 hour period.

EP-A-0 797 991 and WO 99/22724 disclose encapsulated venlafaxine extended release dosage formulation of venlafaxine hydrochloride, which provides in a single dose, a therapeutic blood serum level over a twenty four hour period. Gelatine capsules are filled with film coated spheroids containing venlafaxine hydrochloride. EP-A-0 797 991 states that numerous attempts to produce extended release tablets by hydrogel technology proved to be fruitless because the compressed tablets were either physically unstable (poor compressibility or capping problems) or dissolved too rapidly in dissolution studies. Typically, the tablets prepared as hydrogel sustained release formulations gave 40-50% dissolution at 2 hours, 60-70% dissolution at 4 hours and 85-100% dissolution at 8 hours (EP-A-0 797 991).

WO 94/27589 and WO 01/37815 describe osmotic dosage forms containing venlafaxine hydrochloride.

The object of the invention is to provide an improved solid controlled release pharmaceutical formulation containing venlafaxine and a process for the preparation thereof. This object is achieved for example by the combination of the features in each of the independent claims 1 and 26. Preferable embodiments of the invention are defined in the dependent claims.

The pharmaceutical formulation of the present invention comprises for example a core consisting of an active drug which may be advantageously in amorphous form, polyvinylpyrrolidone, a combination of two hydrophilic polymers having different viscosities and optionally other commonly used ingredients for solid dosage forms. The core is coated with a polymeric coating comprising a combination of two polymers having different water permeabilities. A plasticizer and other commonly used ingredients for film coating may be optionally added thereto.

A solid controlled release formulation according to a preferred embodiment of the present invention comprises for example a core consisting of venlafaxine, polyvinylpyrrolidone, a combination of two different hydrophilic polymers preferably from the group of cellulose ethers from which the first one may be a low viscosity cellulose ether and the second one may be a high viscosity cellulose ether, and other commonly used ingredients for solid dosage forms. The core is coated with a polymeric coating comprising a combination of two different polymers from which the first one is water high permeable polymer and the second one is water low permeable polymer. It is advantageous to further add a plasticizer and other commonly used ingredients for film coating.

Venlafaxine may be in a form of a pharmaceutically acceptable salt, preferably in a form of venlafaxine hydrochloride.

It was unexpected that once daily formulation of venlafaxine hydrochloride could be obtained using hydrogel technology based on a combination of low and high viscosity hydrophilic polymers although the active substance is extremely hydrophilic and water soluble.

Controlled release of venlafaxine hydrochloride over 24 hours is achieved by a combination of two hydrophilic polymers of different viscosity in the core and of two polymers of different water permeability in the coating.

The active ingredient stabilised with polymers is dispersed at the molecular level and has therefore always the same particle size and the same specific surface area. Consequently, the dissolution rate is not polymorph dependent but dependent solely on the combination and ratio of low and high viscosity hydrophilic polymers in the core and on combination and ratio of water high permeable and water low permeable polymers in the coating.

The water soluble polymer polyvinylpyrrolidone prevents the crystallisation of the active ingredient, simultaneously it is a carrier of the active ingredient in the coprecipitate. Polyvinylpyrrolidone with a K-value (relative viscosity of the compound in water solution with regard to water) preferably ranging from 10 to 95, more preferably in the range from 24 to 32, with an average molecular weight preferably ranging from 2000 g/mol to 110000 g/mol, more preferably in the range from 25000 g/mol to 50000 g/mol may be used. Polyvinylpyrrolidone is preferably present in the formulation in the range from 5 to 40 wt %, more preferably from 10 to 20 wt %, with respect to the total weight of the formulation.

The low viscosity hydrophilic polymer acts as a carrier of the active ingredient which simultaneously inhibits its crystallisation in the coprecipitate of venlafaxine hydrochloride and polyvinylpyrrolidone, and together with other ingredients it modifies the release of the active substance in such a way that it is sustained over 24 hour period.

The low viscosity hydrophilic polymer may preferably be present in a quantity from 10 to 70 wt %, more preferably from 20 to 50 wt %, with respect to the total weight of the pharmaceutical formulation.

For providing a stable, preferably amorphous form of the active ingredient in the novel pharmaceutical formulation the required weight ratio between the water soluble polymer polyvinylpyrrolidone and the low viscosity hydrophilic polymer is preferably in the range from 10:1 to 1:10, more preferably in the range from 1:3 to 3:1.

The combination of the carriers i.e. the water soluble polymer polyvinylpyrrolidone and the low viscosity hydrophilic polymer has a double effect and the advantage that it stabilises the amorphous form of the active ingredient and simultaneously modifies the release of the amorphous active ingredient in such a way that it is sustained, repeatable and independent of the amorphous or polymorphous form of the active ingredient, its particle size and specific surface area.

The high viscosity hydrophilic polymer in combination with low viscosity hydrophilic polymer modifies the release of the active substance in such a way that it is sustained over 24 hour period.

High viscosity hydrophilic polymer is present preferably in a quantity from 5 to 70 wt %, more preferably from 7 to 50 wt %, with respect to the total weight of the pharmaceutical formulation.

A low viscosity and a high viscosity hydrophilic polymer can preferably be selected from cellulose ethers selected from the group consisting of methylcellulose, ethylcellulose, hydroxyethylcellulose, propylcellulose, hydroxypropylcellulose, hydroxypropylmethylcellulose, carboxymethylcellulose, preferably hydroxyethylcellulose, hydroxypropylcellulose and hydroxymethylpropylcellulose. Combinations may also be used.

Particularly preferable cellulose ether is hydroxypropylmethylcellulose. A low viscosity hydroxypropylmethylcellulose is defined as one having preferably a molecular weight of 55,000 or less and viscosity of 800 mPas or less. A high viscosity hydroxypropylmethylcellulose is defined as one preferably having a molecular weight of 60,000 or greater and viscosity of 1000 mPas or greater. Different types of hydroxypropylmethylcellulose may be used.

| | | | Relative | |
|---|---|---|---|---|
| | | | rate of | Viscosity |
| | % methoxyl | % hydroxypropoxyl | hydration | mPas |
| Type K | 19-24 | 7-12 | fastest | 3, 100, |
| | | | | 4000, |
| | | | | 15000, |
| | | | | 10000 |
| Type E | 28-30 | 7-12 | next | 3, 5, 6, |
| | | | fastest | 15, 50, |
| | | | | 4000 |
| Type F | 27-30 | 4-7,5 | slower | 50, 4000 |

For providing a sustained release of highly soluble amorphous active ingredient from a novel pharmaceutical formulation over 24 hour period the required ratio between the low viscosity and high viscosity hydrophilic polymer is preferably from 10:1 to 1:3, more preferably from 6:1 to 1:2, in particular preferably from 3:1 to 1:1.

The core may also contain other usual ingredients useful in the preparation of solid pharmaceutical forms such as fillers, binders, swelling excipients, glidants, lubricants etc. The core may contain one or more fillers such as lactose, starch, saccharose, glucose, microcrystalline cellulose, mannitol, sorbitol, calcium hydrogen phosphate, aluminium silicate, sodium chloride. Further it may contain one or more binders such as starch, gelatine, carboxymethylcellulose, polyvinylpyrrolidone, crosslinked polyvinylpyrrolidone, sodium alginate, microcrystalline cellulose etc.; one or-more disintegrants such as starch, cross-linked sodium carboxymethylcellulose, cross-linked polyvinylpyrrolidone, sodium starch glycolate etc., one or more glidants such as magnesium stearate, calcium stearate, aluminium stearate, stearic acid, palmitic acid, cetanol, stearol, polyethylene glycols of various molecular weights, talc, etc., one or more lubricants such as stearic acid, calcium, magnesium or aluminium stearate, siliconized talc etc.

The formulation contains in a preferred embodiment from 10 to 400 mg of venlafaxine, more preferably from 30 to 200 mg of venlafaxine, particularly preferably from 37,5 to 150 mg of venlafaxine. venlafaxine is in a form of pharmaceutically acceptable salt, more preferably as venlafaxine hydrochloride.

The film coating comprises a combination of two different polymers from which the first one is a water high permeable polymer and the second one is a water low permeable polymer.

As a water high permeable polymers are considered polymers which are soluble (suitably 3.3% or more, more suitably 5% or more, even more suitably 10% or more, particularly suitably 50% or more and especially suitably 70% or more ) in water (e.g. hydroxypropylcellulose, hydroxypropylmethylcellulose, methylcellulose and hydroxyethylcellulose) or can achieve water permeability by swelling or salt formation (e.g. methacrylate aminoester copolymer, methylcellulose) or contain groups permeable for water in a high proportion (suitably molar ratio of water permeable to water non-permeable groups is 1:30 or more) (e.g. high permeable poly(ethylacrylate, methylmethacrylate)trimethylammoniumethylmethacrylate chloride).

As water low permeable polymers are considered polymers which are insoluble in water, some in entire physiological pH (e.g. ethylcellulose) and some in acidic pH (e.g. cellulose acetate phthalate, methacrylic acid copolymers, polyvinyl acetate phthalate, cellulose acetate trimellitate, hydroxypropylmethylcellulose phthalate), or contain groups permeable for water in a small proportion (suitably molar ratio of water permeable to water non-permeable groups is 1:30 or less) (e.g. low permeable poly(ethylacrylate, methylmethacrylate)trimethylammoniumethylmethacrylate chloride).

Water high permeable polymer may preferably be selected from methylcellulose, hydroxypropylcellulose, hydroxypropylmethylcellulose, hydroxyethylcellulose, methacrylate aminoester copolymer, high permeable poly(ethylacrylate, methylmethacrylate)trimethylammoniumethylmethacrylate chloride, preferably hydroxypropylcellulose, hydroxypropylmethylcellulose, methylcellulose and high permeable poly(ethylacrylate, methylmethacrylate)trimethylammoniumethylmethacrylate chloride, most preferably from hydroxypropylcellulose, hydroxypropylmethylcellulose and high permeable poly(ethylacrylate, methylmethacrylate)trimethylammoniumethylmethacrylate chloride. The selection of the water high permeable polymer should not be restricted by these examples.

Water low permeable polymer may preferably be selected from ethylcellulose, cellulose acetate phthalate, methacrylic acid copolymers, polyvinyl acetate phthalate, cellulose acetate trimellitate, hydroxypropylmethylcellulose phthalate and low permeable poly(ethylacrylate, methylmethacrylate)trimethylammoniumethylmethacrylate chloride, preferably ethylcellulose, hydroxypropylmethylcellulose phthalate, polyvinyl acetate phthalate and low permeable poly(ethylacrylate, methylmethacrylate)trimethylammoniumethylmethacrylate chloride, most preferably from ethylcellulose, hydroxypropylmethylcellulose phthalate and low permeable poly(ethylacrylate, methylmethacrylate)trimethylammoniumethylmethacrylate chloride. The selection of the water low permeable polymer should not be restricted by these examples.

The combinations of water high permeable and water low permeable polymers may be selected in particular from, but not limited to, combination of hydroxypropylmethylcellulose and hydroxypropylmethylcellulose phthalate, hydroxypropylcellulose and hydroxypropylmethylcellulose phthalate, hydroxypropylmethylcellulose and ethylcellulose, hydroxypropylcellulose and ethylcellulose, hydroxypropylmethylcellulose and polyvinyl acetate phthalate, hydroxypropylcellulose and polyvinyl acetate phthalate, high permeable poly(ethylacrylate, methylmethacrylate)trimethylammoniumethylmethacrylate chloride and low permeable poly(ethylacrylate, methylmethacrylate)trimethylammoniumethylmethacrylate chloride, preferably hydroxypropylmethylcellulose and hydroxypropylmethylcellulose phthalate, hydroxypropylcellulose and ethylcellulose, hydroxypropylmethylcellulose and ethylcellulose, high permeable poly(ethylacrylate, methylmethacrylate)trimethylammoniumethylmethacrylate chloride and low permeable poly(ethylacrylate, methylmethacrylate)trimethylammoniumethylmethacrylate chloride.

The ratio between the water high permeable and water low permeable polymers is preferably from 10:1 to 1:5, more preferably from 6:1 to 1:4, particularly preferably from 3:1 to 1:3.

The coating is preferably present in the formulation in the range from 1 to 15 wt %, more preferably from 2 to 10 wt %, with respect to the total weight of the formulation.

The coating may also contain other usual ingredients useful in the preparation of film coated solid dosage forms such as plasticizers, fillers, antisticking agents, antifoams, colorants etc.

The coating may contain one or more plasticizers such acetyl tributyl citrate, acetyl thriethyl citrate, acetylated fatty acid glycerides, castor oil, dibutyl phthalate, diethyl phthalate, diethyl sebacate, dibutyl sebacate, dimethyl phthalate, glycerol, glycerol monostearate, glycelyl triacetate, polyethylene glycols, polyoxyethylene/polyoxypropylene copolymers, propylene glycol, tributyl citrate, triethyl citrate. Further it may contain one or more fillers such as lactose, polydextrose and maltodextrin; one or more antisticking agents such as talc, magnesium stearate, calcium stearate, etc., one or more antifoams such as dimethylpolysiloxane, etc., one or more colorants such as titanium dioxide, iron oxides, lakes, etc.

A combination of amorphous form of an active ingredient in the coprecipitate with water soluble polyvinylpyrrolidone and a combination of low viscosity and high viscosity hydrophilic polymer in the core and a combination of water high permeable and water low permeable polymer in the coating, prepared in a certain ratio between the single components of the formulation according to the process of the invention, which is simple and technologically as well as economically acceptable, has hitherto not been described in the literature. The granulation of an active ingredient, the water soluble polymer polyvinylpyrrolidone and a combination of low viscosity and high viscosity hydrophilic polymer and other ingredients suitable for preparation of solid pharmaceutical forms has good compressibility, so prepared tablets are firm, have low friability and together with a combination of water high permeable and water low permeable polymer in the coating make possible a sustained release of the amorphous active ingredient from pharmaceutical formulation over 24 hour period. Due to the preferable amorphous form of the active ingredient, the release rate of the active ingredient is not dependent on polymorphic form and particle size of active ingredient but solely on the combination and ratio of low and high viscosity hydrophilic polymers in the core and on combination and ratio of water high permeable and water low permeable polymer in the coating.

The above object can also be achieved by a process defined in claim 26. For example in the first step of the preparation of a pharmaceutical formulation according to the invention an active ingredient and the water soluble polymer polyvinylpyrrolidone are dissolved in an organic solvent at a temperature e.g. from 20 to 60° C., and preferably in a fluid bed granulator. The obtained solution is applyed, preferably sprayed onto a low viscosity hydrophilic polymer such as e.g. cellulose ether in the fluid bed. As the active ingredient there can be used an amorphous form or a polymorphous form of the active ingredient which in the process of coprecipitation according to the invention is converted into an amorphous form stabilised with water soluble polyvinylpyrrolidone and low viscosity hydrophilic polymer. Organic solvents useful for this purpose can be selected from group of alcohols, ketones, esters, aliphatic hydrocarbons, halogenated hydrocarbons, cycloaliphatic, aromatic, heterocyclic solvents or mixtures thereof. Typical solvents can be ethanol, methanol, isopropyl alcohol, n-butyl alcohol, acetone, diethyl ether, ethyl acetate, isopropyl acetate, methyl acetate, dichloromethane, chloroform, mixtures of these solvents such as ethanol and acetone, methanol and acetone, dichloromethane and methanol and mixtures thereof. If a polymorphous form of the active ingredient is chosen, it is in the process of the invention converted into an amorphous form which is stabilised with water soluble polymer polyvinylpyrrolidone and low viscosity hydrophilic polymer. The obtained granulation is suitably regranulated through a sieve of mesh size 0.5 mm at room temperature.

The second step of the preparation of a pharmaceutical formulation according to the invention is, for example, conducted in such a manner that at room temperature the granulation obtained in the first step is homogeneously blended with a high viscosity hydrophilic polymer and other usual adjuvants useful in the preparation of solid pharmaceutical forms such as lactose, polyvinylpyrrolidone, cross-linked polyvinylpyrrolidone, starch, calcium hydrogen phosphate, calcium hydrogen carbonate, aluminium silicate, magnesium stearate, talc, or generally with fillers, binders, disintegrants, glidants, lubricants etc.

The components are compressed to obtain a core which may suitably be provided as tablets obtainable with known tableting machines. Thus it is possible to prepare tablets with controlled release of an amorphous active ingredient in a relatively simple and economical way.

In the third step of preparation of a pharmaceutical formulation according to the invention the obtained cores are, for example, film coated with a combination of water high permeable and water low permeable polymer. The coating can be performed using dispersion or colloidal solution. Colloidal solution is prepared by dissolving the polymers e.g. in an organic solvent, in mixtures of organic solvents or in mixtures thereof with water. As organic solvent ethanol, methanol, propan-2-ol, acetone, ethyl acetate, glacial acetic acid, glycols, dichloromethane, dimethyl formamide, dimethylsulfoxide, dioxane chloroform, toluene, methylene chloride, benzene, diaceton alcohol, ethoxyethyl acetate, ethylene glycol monoacetate, ethyl lactate, methoxyethyl acetate, β-methoxyethylene alcohol, methylethyl ketone can be used.

Coating dispersion can be prepared either by mixing powders of polymers or other suitable ingredients in organic solvent or in combination of organic solvent with water or by mixing and diluting aqueous dispersion of polymers with water.

In the second step plasticizer or a mixture of plasticizers may be optionally added to the polymer colloidal solution or dispersion of polymers and then the suspension of colorants, antisticking agents, fillers, antifoams may be added. The coating can be performed by means of known coating techniques in perforated coating pans. Thus it is possible to prepare film coated tablets with sustained release of an amorphous active ingredient in a relatively simple and economical way.

The invention is illustrated, but in no way limited by the following examples:

### EXAMPLE 1

Composition of one tablet

| Core: | | |
|---|---|---|
| Venlafaxine hydrochloride | 150 | mg |
| polyvinylpyrrolidone K30 | 150 | mg |
| Hydroxypropylmethylcellulose Methocel F50P | 450 | mg |
| Hydroxypropylmethylcellulose Methocel K100MP | 70 | mg |
| Ludipress | 173 | mg |
| Talc | 5 | mg |
| Mg stearate | 2 | mg |
| Coating: | | |
| Hydroxypropylmethylcellulose Pharmacoat 606 | 22,695 | mg |
| Hydroxypropylmethylcellulose phthalate | 9,726 | mg |
| Triethyl citrate | 2,598 | mg |
| Iron oxide yellow | 0,788 | mg |
| Titanium dioxide | 2,373 | mg |
| Talc | 0,324 | mg |

A batch of 800 tablets was prepared according to the following procedure:

Crystalline venlafaxine hydrochloride (120 g) and polyvinylpyrrolidone K30 (120 g) (Kollidon 30, BASF; Plasdone K-30, ISP GAF) were dissolved in ethanol (960 g) under intensive stirring at room temperature. The formed solution (1200 g) was in a fluid bed at an inlet air temperature from 70° C. to 85° C. sprayed onto hydroxypropylmethylcellulose (360 g) with a viscosity of 50 mPas (Methocel F50 premium, Dow Chemicals) and with an average molecular weight of 19000 g/mol. The so prepared granulation (600 g) was dried in a fluid bed and regranulated through a sieve with mesh size 0.5 mm. To the granulation there were added hydroxypropylmethylcellulose (56 g) with a viscosity of 100000 mPas (Methocel K100M premium, Dow Chemicals) and with an average molecular weight of 215000 g/mol, Ludipress (138.4 g) (93.4 wt % of lactose monohydrate+3.2 wt % of polyvinylpyrrolidone K30 (Kollidon 30)+3.4 wt % of cross-linked polyvinylpyrrolidone (Kollidon CL, BASF Germany)), talc (4 g) and magnesium stearate (1.6 g) and they were homogeneously blended at room temperature. The so prepared granulation with amorphous venlafaxine hydrochloride was compressed into tablets using usual tableting machine so that tablets with a weight of 950 mg were obtained.

Hydroxypropylmethylcellulose (36.312 g) (Pharmacoat 606, Shin Etsu Chemical Co. Japan) with a viscosity of 6 mPas, Hydroxypropylmethylcellulose phthalate (15.562 g) (HP-50, Shin Etsu Chemical Co. Japan ) and triethyl citrate (4.150 g) (Morflex) were dissolved while stirring in a mixture of ethanol (410.774 g) and water (153.742 g), and then homogenised (Ultraturax, 30 min.) suspension of titanium dioxide (3.797 g), iron oxide yellow (1.261 g) (Sicopharm 10, BASF) and talcum (0.518 g) in water (22.304 g) was added. Prepared suspension was sprayed onto cores so that the film coating in a weight ratio of about 4.8 wt. % regard to core was obtained. Tablets were also polished with talcum (0.563 g).

Dissolution of the tablets.

| Apparatus: | | apparatus 2 (USP 23), 100 rpm |
|---|---|---|
| Medium: | 0-2 hours: | artificial gastric juice pH 1.2 |
| | 2-24 hours: | artificial intestinal juice pH 6.8 |

| | |
|---|---|
| Temperature: | 37° C. |
| Quantitative analysis: | UV spectrophotometry, 273 nm |

TABLE 1
Percentage of released venlafaxine vs. dissolution time

| Dissolution time | Percentage of released |
|---|---|
| (h) | venlafaxine |
| 1 | 13.2 |
| 2 | 22.0 |
| 3 | 32.1 |
| 4 | 39.6 |
| 6 | 51.3 |
| 8 | 60.7 |
| 10 | 68.5 |
| 12 | 74.9 |
| 14 | 80.3 |
| 16 | 84.6 |
| 18 | 88.2 |
| 20 | 91.9 |
| 22 | 93.4 |
| 24 | 95.5 |

From the above table it is evident that venlafaxine dissolution is sustained over 24 hour period.

### EXAMPLE 2

Composition of one tablet

| Core: | | |
|---|---|---|
| Venlafaxine hydrochloride | 169,73 | mg |
| Polyvinylpyrrolidone K30 | 150 | mg |
| Hydroxypropylmethylcellulose Methocel F50P | 380,27 | mg |
| Hydroxypropylmethylcellulose Methocel K100MP | 150 | mg |
| Ludipress | 93 | mg |
| Talc | 5 | mg |
| Mg stearate | 2 | mg |
| Coating: | | |
| Hydroxypropylcellulose Klucel EF | 7,620 | mg |
| Ethylcellulose N7 | 17,780 | mg |
| Triethyl citrate | 2,298 | mg |
| Titanium dioxide | 7,756 | mg |
| Talc | 2,546 | mg |

A batch of 800 tablets was prepared according to the following procedure: Tablet cores were prepared according to the same procedure as in Example 1.

Hydroxypropylcellulose (12.192 g) (Klucel EF, Hercules, Wilmington) with an average molecular weight of 60000 g/mol and a viscosity of 5-10 mPas , Ethylcellulose (28.448 g) (N7, Hercules, Wilmington) ethoxyl content 48.0-49.5% and viscosity 5.6-8 mPas and triethyl citrate (3.677 g) (Morflex) were dissolved while stirring in ethanol (548.823 g), and then homogenised (Ultraturax, 30 min.) suspension of titanium dioxide (12.410 g) and talcum (4.073 g) in ethanol (65.932 g) was added. Prepared suspension was sprayed onto cores so that the film coating in a weight ratio of about 4.0 wt. % regard to the core was obtained. Tablets were also polished with talcum (0.563 g).

Dissolution of the tablets.

| Apparatus: | | apparatus 2 (USP 23), 150 rpm |
|---|---|---|
| Medium: | 0-2 hours: | artificial gastric juice pH 1.2 |
| | 2-24 hours: | artificial intestinal juice pH 6.8 |

| | |
|---|---|
| Temperature: | 37° C. |
| Quantitative analysis: | UV spectrophotometry, 273 nm |

TABLE 1
Percentage of released venlafaxine vs. dissolution time

| Dissolution time | Percentage of released |
|---|---|
| (h) | venlafaxine |
| 0.5 | 1.0 |
| 1 | 4.4 |
| 1.5 | 8.4 |
| 2 | 12.4 |
| 3 | 21.1 |
| 4 | 28.0 |
| 6 | 40.0 |
| 8 | 50.1 |
| 10 | 58.8 |
| 12 | 66.6 |
| 16 | 78.8 |
| 18 | 83.3 |
| 20 | 87.1 |
| 24 | 92.0 |

From the above table it is evident that venlafaxine dissolution is sustained over 24 hour period.

### EXAMPLE 3

Composition of one tablet

| Core: | | |
|---|---|---|
| Venlafaxine hydrochloride | 169,73 | mg |
| Polyvinylpyrrolidone K30 | 150 | mg |
| Hydroxypropylmethylcellulose Methocel F50P | 380,27 | mg |
| Hydroxypropylmethylcellulose Methocel K100MP | 150 | mg |
| Ludipress | 93 | mg |
| Talc | 5 | mg |
| Mg stearate | 2 | mg |
| Coating: | | |
| Hydroxypropylcellulose Klucel EF | 10,160 | mg |
| Ethylcellulose N7 | 15,240 | mg |
| Triethyl citrate | 2,298 | mg |
| Titanium dioxide | 7,756 | mg |
| Talc | 2,546 | mg |

A batch of 800 tablets was prepared according to the following procedure: Tablet cores were prepared according to the same procedure as in Example 1.

The coating of the tablet cores was performed according to the same procedure as an example 2 only a ratio of Hydroxypropylcellulose to Ethylcellulose 1:1.5 was used.

Dissolution of the tablets.

| Apparatus: | | apparatus 2 (USP 23), 150 rpm |
|---|---|---|
| Medium: | 0-2 hours: | artificial gastric juice pH 1.2 |
| | 2-24 hours: | artificial intestinal juice pH 6.8 |

| | |
|---|---|
| Temperature: | 37° C. |
| Quantitative analysis: | UV spectrophotometry, 273 nm |

TABLE 1
Percentage of released venlafaxine vs. dissolution time

| Dissolution time | Percentage of released |
|---|---|
| (h) | venlafaxine |
| 0.5 | 5.8 |
| 1 | 11.8 |
| 1.5 | 16.9 |
| 2 | 21.1 |
| 3 | 29.9 |
| 4 | 37.0 |
| 6 | 48.8 |
| 8 | 58.7 |
| 10 | 67.0 |
| 12 | 74.2 |
| 16 | 84.7 |
| 18 | 88.2 |
| 20 | 91.0 |
| 24 | 94.1 |

### EXAMPLE 4

Composition of one tablet

| Core: | | |
|---|---|---|
| Venlafaxine hydrochloride | 169,73 | mg |
| Polyvinylpyrrolidone K30 | 150 | mg |
| Hydroxypropylmethylcellulose Methocel F50P | 380,27 | mg |
| Hydroxypropylmethylcellulose Methocel K100MP | 250 | mg |
| Ludipress | 93 | mg |
| Talc | 5 | mg |
| Mg stearate | 2 | mg |
| Coating: | | |
| Eudragit RL30D | 2,450 | mg |
| Eudragit RS30D | 1,050 | mg |
| Triethyl citrate | 0,700 | mg |
| Titanium dioxide | 3,815 | mg |
| Talc | 5,404 | mg |
| Polyethylene glycol 6000 | 0,318 | mg |
| Polydimethylsiloxane | 0,032 | mg |

A batch of 800 tablets was prepared according to the following procedure: Tablet cores were prepared according to the same procedure as in Example 1.

The coating of the tablet cores was performed according to the following procedure:

In the first step, polyethylene glycol (2.289 g) with a molecular weight of 5400-6600 (Clariant) was dissolved in part of the water (4.647 g). This solution and talcum (38.910 g), titanium dioxide (27.468 g), dimethylpolysiloxane (0.231 g) (Merck) were then stirred into part of the water (155.577 g) and homogenised (Ultraturrax, 30 min.). To eliminate air bubbles stirring of pigment suspension was continued overnight (approx. 12 hours).

The polymer dispersion were prepared from Eudragit RL 30D (58.800 g 30% aqueous dispersion of poly (ethylacrylate, methylmethacrylate) trimethylammoniumethylmethacrylate chloride), Eudragit RS 30D (25.200 g 30% aqueous dispersion of poly(ethylacrylate, methylmethacrylate)trimethylammoniumethylmethacrylate chloride), triethyl citrate (5.040 g) and water (139.620 g) while mixing for 30 min.

The pigment suspension and polymer dispersion were mixed for 20 min shortly before use.

So prepared suspension was sprayed onto cores so that the film coating in a weight ratio of about 1.3 wt. % regard to the core was obtained.

Dissolution of the tablets.

| Apparatus: | | apparatus 2 (USP 23), 150 rpm |
|---|---|---|
| Medium: | 0-2 hours: | artificial gastric juice pH 1.2 |
| | 2-24 hours: | artificial intestinal juice pH 6.8 |

| | |
|---|---|
| Temperature: | 37° C. |
| Quantitative analysis: | UV spectrophotometry, 273 nm |

TABLE 1
Percentage of released venlafaxine vs. dissolution time

| Dissolution time | Percentage of released |
|---|---|
| (h) | venlafaxine |
| 0.5 | 10.0 |
| 1 | 15.0 |
| 1.5 | 19.4 |
| 2 | 23.1 |
| 3 | 31.2 |
| 4 | 37.5 |
| 6 | 48.1 |
| 8 | 56.8 |
| 10 | 64.2 |
| 12 | 70.0 |
| 16 | 81.2 |
| 18 | 85.1 |
| 20 | 88.2 |
| 24 | 92.7 |

From the above table it is evident that venlafaxine dissolution is sustained over 24 hour period.

### EXAMPLE 5

Composition of one tablet

| Core: | | |
|---|---|---|
| Venlafaxine hydrochloride | 169,73 | mg |
| Polyvinylpyrrolidone K30 | 150 | mg |
| Hydroxypropylmethylcellulose Methocel F50P | 380,27 | mg |
| Hydroxypropylmethylcellulose Methocel K100MP | 250 | mg |
| Ludipress | 93 | mg |
| Talc | 5 | mg |
| Mg stearate | 2 | mg |
| Coating: | | |
| Hydroxypropylcellulose Klucel EF | 12,700 | mg |
| Ethylcellulose N7 | 12,700 | mg |
| Triethyl citrate | 2,298 | mg |
| Titanium dioxide | 7,756 | mg |
| Talc | 2,546 | mg |

A batch of 800 tablets was prepared according to the following procedure: Tablet cores were prepared according to the same procedure as in Example 1.

The coating of the tablet cores was performed according to the same procedure as an example 2 only a ratio of hydroxypropylcellulose to ethylcellulose 1:1 was used.

Dissolution of the tablets.

| Apparatus: | | apparatus 2 (USP 23), 150 rpm |
|---|---|---|
| Medium: | 0-2 hours: | artificial gastric juice pH 1.2 |
| | 2-24 hours: | artificial intestinal juice pH 6.8 |

| | |
|---|---|
| Temperature: | 37° C. |
| Quantitative analysis: | UV spectrophotometry, 273 nm |

TABLE 1
Percentage of released venlafaxine vs. dissolution time

| Dissolution time | Percentage of released |
|---|---|
| (h) | venlafaxine |
| 0.5 | 8.2 |
| 1 | 13.5 |
| 1.5 | 18.1 |
| 2 | 22.0 |
| 3 | 30.6 |
| 4 | 37.3 |
| 6 | 48.5 |
| 8 | 57.9 |
| 10 | 65.7 |
| 12 | 72.6 |
| 16 | 83.3 |
| 18 | 87.3 |
| 20 | 90.4 |
| 24 | 94.2 |

From the above table it is evident that venlafaxine dissolution is sustained over 24 hour period.

1. A solid controlled release pharmaceutical formulation for once daily administration comprising
a core comprising venlafaxine, polyvinylpyrrolidone, a low viscosity hydrophilic polymer and a high viscosity hydrophilic polymer; and
a polymeric coating comprising a water high permeable polymer, and a water low permeable polymer.

2. The formulation of claim 1 wherein venlafaxine is in a form of venlafaxine hydrochloride.

3. The formulation of claim 1 wherein venlafaxine is in amorphous form.

4. The formulation of claim 1 wherein the amount of venlafaxine is from 10 to 400 mg.

5. The formulation of claim 1 wherein the amount of venlafaxine is from 37.5 to 150 mg.

6. The formulation of claim 1.wherein the amount of venlafaxine is 75 mg.

7. The formulation of claim 1 wherein the amount of venlafaxine is 150 mg.

8. The formulation of claim 1 wherein the low viscosity hydrophilic polymer and the high viscosity hydrophilic polymer in the core are selected from cellulose ethers.

9. The formulation of claim 1 wherein the low viscosity hydrophilic polymer and the high viscosity hydrophilic polymer in the core are selected from the group consisting of methylcellulose, ethylcellulose, hydroxyethylcellulose, propylcellulose, hydroxypropylcellulose, hydroxypropylmethylcellulose or carboxymethylcellulose.

10. The formulation of claim 1 wherein the low viscosity hydrophilic polymer and the high viscosity hydrophilic polymer in the core are a low viscosity hydroxypropylmethylcellulose and a high viscosity hydroxypropylmethylcellulose.

11. The formulation of claim 1 wherein the ratio between polyvinylpyrrolidone and the low viscosity hydrophilic polymer in the core is from 10:1 to 1:10.

12. The formulation of claim 1 wherein the ratio between polyvinylpyrrolidone and the low viscosity hydrophilic polymer in the core is from 1:3 to 3:1.

13. The formulation of claim 1 wherein the ratio between the low viscosity hydrophilic polymer and the high viscosity hydrophilic polymer in the core is from 10:1 to 1:3.

14. The formulation of claim 1 wherein the ratio between the low viscosity hydrophilic polymer and the high viscosity hydrophilic polymer in the core is from 3:1 to 1:1

15. The formulation of claim 1 wherein the water high permeable polymer in the coating is selected from the group consisting of methylcellulose, hydroxypropylcellulose, hydroxypropylmethylcellulose, hydroxyethylcellulose, methacrylate aminoester copolymer, high permeable poly(ethylacrylate, methylmethacrylate)trimethylammoniumethylmethacrylate chloride.

16. The formulation of claim 1 wherein the water high permeable polymer in the coating is selected from the group consisting of hydroxypropylcellulose, hydroxypropylmethylcellulose and high permeable poly(ethylacrylate, methylmethacrylate)trimethylammoniumethylmethacrylate chloride.

17. The formulation of claim 1 wherein the water low permeable polymer in the coating is selected from ethylcellulose, cellulose acetate phthalate, methacrylic acid copolymers, polyvinyl acetate phthalate, cellulose acetate trimellitate, hydroxypropylmethylcellulose phthalate and low permeable poly(ethylacrylate, methylmethacrylate)trimethylammoniumethylmethacrylate chloride.

18. The formulation of claim 1 wherein the water low permeable polymer in the coating is selected from ethylcellulose, hydroxypropylmethylcellulose phthalate and low permeable poly(ethylacrylate, methylmethacrylate)trimethylammoniumethylmethacrylate chloride.

19. The formulation of claim 1 wherein the combinations of water high permeable and water low permeable polymers is selected from: hydroxypropylmethylcellulose and hydroxypropylmethylcellulose phthalate, hydroxypropylcellulose and hydroxypropylmethylcellulose phthalate, hydroxypropylmethylcellulose and ethylcellulose, hydroxypropylcellulose and ethylcellulose, hydroxypropylmethylcellulose and polyvinyl acetate phthalate, hydroxypropylcellulose and polyvinyl acetate phthalate, high permeable poly(ethylacrylate, methylmethacrylate)trimethylammoniumethylmethacrylate chloride and low permeable poly(ethylacrylate, methylmethacrylate)trimethylammoniumethylmethacrylate chloride.

20. The formulation of claim 1 wherein the combination of water high permeable and water low permeable polymers is selected from the following combinations: hydroxypropylmethylcellulose and hydroxypropylmethylcellulose phthalate, hydroxypropylcellulose and ethylcellulose, hydroxypropylmethylcellulose and ethylcellulose, high permeable poly(ethylacrylate, methylmethacrylate)trimethylammoniumethylmethacrylate chloride and low permeable poly(ethylacrylate, methylmethacrylate)trimethylammoniumethylmethacrylate chloride.

21. The formulation of claim 1 wherein the ratio between the water high permeable and the water low permeable polymer in the coating is from 10:1 to 1:5.

22. The formulation of claim 1 wherein the ratio between the water high permeable and the water low permeable polymers in the coating is from 3:1 to 1:3

23. The formulation of claim 1 wherein the coating represents 1 to 15 wt. % of the total weight of the formulation.

24. The formulation of claim 1 wherein the coating further comprises a plasticizer selected from the group consisting of acetyl tributyl citrate, acetyl thriethyl citrate, acetylated fatty acid glycerides, castor oil, dibutyl phthalate, diethyl phthalate, diethyl sebacate, dibutyl sebacate, dimethyl phthalate, glycerol, glycerol monostearate, glycelyl triacetate, polyethylene glycols, polyoxyethylene/polyoxypropylene copolymers, propylene glycol, tributyl citrate, triethyl citrate.

25. The formulation of claim 24 wherein the plasticizer is triethyl citrate.

26. A process for the preparation of a solid controlled release pharmaceutical formulation comprising the steps of:
dissolving venlafaxine and polyvinylpyrrolidone in an organic solvent,
applying the resulting solution onto low viscosity polymer,
homogeneously mixing the obtained granulate with a high viscosity polymer, and
compressing the granulate to obtain a core which is then coated with a polymeric coating comprising a water high permeable polymer and a water low permeable polymer.

27. The process of claim 26 wherein venlafaxine in amorphous or in polymorphous form is dissolved in an organic solvent.

28. The process of claim 27 wherein the organic solvent is selected from the group consisting of ethanol, methanol, isopropyl alcohol, n-butyl alcohol, acetone, diethyl ether, ethyl acetate, isopropyl acetate, methyl acetate, dichloromethane, chloroform and mixtures thereof.

29. The process of claim 28 wherein the organic solvent is ethanol.

30. The process of claim 26 wherein the film coating is applied from organic solvent or aqueous media.

31-37. (canceled)
